(19) United States
(12) Patent Application Publication
(10) Pub. No.: US 2009/0285920 A1
(43) Pub. Date: Nov. 19, 2009

(54) NATURAL ANTIBIOTICS CONTAINING A FERMENTATION PRODUCT OF GARLIC BY LACTOBACILLI

(75) Inventors: Jong-Hwa LEE, Anseong-shi (KR); Ji-Hyoung Woo, Cheonan-shi (KR); Ah-Rum Lee, Daegu (KR); Hyo-Bin Lim, Cheonan-shi (KR); Jung-Sook Lee, Daejeon (KR); Jong-Seog Ahn, Daejeon (KR)

Correspondence Address: LEXYOUME IP GROUP, LLC, 5180 PARKSTONE DRIVE, SUITE 175, CHANTILLY, VA, 20151, US

(73) Assignees: Liisna Inc., Cheonyan-city (KR); KOREA RESEARCH INSTITUTE OF BIOSCIENCE AND BIOTECHNOLOGY, Daejeon-city (KR)

(21) Appl. No.: 12/199,147

(22) Filed: Aug. 27, 2008

(30) Foreign Application Priority Data
May 15, 2008 (KR) 10-2008-0045080

(51) Int. Cl. A61K 36/8962; A61P 31/04

(52) U.S. Cl. 424/754

(57) ABSTRACT

The present invention relates to an antibacterial composition including a fermented garlic solution that is fermented by lactic acid bacteria. More specifically, it provides an antibacterial substance active against gram-positive and gram-negative bacteria, yeast, and antibiotic-resistant bacteria. An antibacterial composition according to the present invention that includes a fermented garlic solution that is fermented by lactic acid bacteria as an active ingredient controls the growth of bacteria or fungi, and is thereby able to prevent and treat infections thereof.

### CROSS-REFERENCE TO RELATED APPLICATION

This application claims priority to and the benefit of Korean Patent Application No. 10-2008-0045080 filed in the Korean Intellectual Property Office on May 15, 2008, the entire contents of which are incorporated herein by reference.

### BACKGROUND OF THE INVENTION

(a) Field of the Invention

A novel and efficient substance with antibacterial activity is provided. More specifically, an antibacterial substance that has antibacterial effects against gram-positive and gram-negative bacteria, yeast, and antibiotic-resistant bacteria, and that is capable of replacing conventionally used antibiotics, is provided.

(b) Description of the Related Art

Since 6000 BC humans have produced beer using yeast, cheese using fungi, and vinegar using acetobacter. Koreans too have used lactic acid bacteria to make Kimchi. As such, humans have used fungi and various bacteria for fermentation from before the term “fermentation” was first used. There are many benefits provided by fermentation, and Kimchi has proved its virtue to the extent that it is praised as one of the top five healthiest foods in the world.

Garlic, a major ingredient of Kimchi, has recently been in the spotlight as a healthy food ingredient. Sulfur compounds in garlic, such as alin, allicin, diallyl disulfide, diallyl trisulfide, and S-adenyl cysteine are known to work to prevent many diseases and to exhibit a strong antibacterial activity, as confirmed by a number of studies. To enhance garlic's function, studies are currently being performed to isolate components in garlic through processes such as extraction, distillation, natural ripening, and like.

However, the reason why Kimchi, not garlic itself, is one of the top five healthiest foods is not that a special garlic component is somehow extracted, distilled, or ripened and comes to exist in vegetables into which garlic is added. Instead, secondary metabolites, which are not present in the original ingredient and rather are produced through the fermentation process by lactic acid bacteria, are considered to contribute to the antimicrobial activity and anti-cancer effect of Kimchi. Nonetheless, no microorganism has been discovered to have the ability to ferment garlic, which possesses not only the antibacterial activity but also toxicity. Hence, no known study on garlic has discussed fermentation of garlic.

Meanwhile, antibiotics in the livestock industry are commonly used for the purposes of treating infectious diseases, but at the same time are applied to prevent disease and promote animals' growth. In fact, farm animals are raised in small areas at a high density and are exposed to great possibility of disease infection. As such, even with no signal of disease noticed, antibiotics are often used to prevent disease. In addition, antibiotics are used to promote animals' growth as productivity is observed to increase by the use of antibiotics. Antibiotic-resistance is a natural symptom existing in antibiotic use. However, rampant use of antibiotics when not exposed to any diseases or pathogens results in an accelerated process of antibiotic resistance and may even cause serious consequences to public health. High levels of antibiotic resistance may cause a worse situation where clinical treatments against infections diseases do not work.

It is reported that the amount of antibiotics used for animals in Korea is far higher than in other developed countries such as the United States and Japan. A document prepared by the Korea Food and Drug Administration submitted to the Health and Human Service Council in the National Assembly of Korea reports that, as of 2004, a total of 1368 tons of antibiotics were used for the total meat production of 1493 thousand tons, meaning that the ratio of antibiotics used per thousand units of meat production was 0.916. This value is higher than those in the United States (0.254, Korea's being 3.6 times the amount), Japan (0.355, Korea's being 2.6 times the amount), and Australia (0.063, Korea's being 14.5 times the amount).

Taking the example of oxytetracycline (74% market share), its resistance rate is reported to be above 80% following its widespread use for a long time (National Safety Management Project for Antibiotics Resistance, 2003). The WHO recommends not using human antibiotics for animals and is making efforts to replace antibiotic use while providing animals with improved living conditions such as hygienic areas, clean water, and high quality foods. The European Union has banned applying human antibiotics to animals. In Korea as well, a total ban against use of animal antibiotics is expected by 2012. In fact, after avoparcin—an animal antibiotic used for animal growth promotion—was banned in Germany and Denmark, resistance rates for animals and humans were observed to decrease. While there are an increasing number of Korean farms that have adopted a “no antibiotics policy” in pig farming, the difficulties involved in the policy have yet to be resolved with regard to facility investment and profitability issues in order for the policy to become popular.

Therefore there is a strong need for urgent development of a natural composition that is safe to apply and has antibacterial activity, particularly against bacteria with antibiotic resistance to currently available antibiotics.

### SUMMARY OF THE INVENTION

Therefore, an object of an embodiment of the present invention is to provide a novel technology to prevent pathological bacteria.

More specifically, an embodiment of the present invention provides a natural antibacterial agent containing one or more selected from the group consisting of a fermented garlic solution fermented by using lactic acid bacteria, a concentrate of the same, a dried substance of the same, and a combination thereof.

Another embodiment of the present invention provides a composition for preventing fungi or bacterial infection, containing a natural antibacterial agent as an active ingredient, wherein the natural antibacterial agent contains one or more selected from the group consisting of a fermented garlic solution fermented by lactic acid bacteria, a concentrate of the same, a dried substance of the same, and a combination thereof.

Another embodiment of the present invention provides a method for preventing fungi or bacterial infection by using a natural antibacterial agent containing one or more selected from the group consisting of a fermented garlic solution fermented by lactic acid bacteria, a concentrate of the same, a dried substance of the same, and a combination thereof.

### BRIEF DESCRIPTION OF THE DRAWINGS

FIG. 1 is a graph showing survival rates of MDCK cells in different concentrations of garlic broth.

FIG. 2. is a graph showing survival rates of MDCK cells in different concentrations of fermented garlic solution.

### DETAILED DESCRIPTION OF THE EMBODIMENTS

Although it is well-known that garlic exerts antibacterial and anti-cancer effects, no microorganisms have been found that are capable of fermenting garlic and that not only have an antibacterial property but also toxicity. Therefore, few studies on fermentation products of garlic have been made. The present inventors observed, however, that such problems could be overcome by lactic acid bacteria that ferments garlic in Kimchi. The present inventors found that an excellent antibacterial agent can be produced by fermenting garlic with lactic acid bacteria in Kimchi, to complete the present invention. It is thus expected that antibacterial substances according to the present invention can replace currently used antibiotics that involve resistance problems.

An embodiment of the present invention provides a natural antibacterial agent that includes, as an active ingredient, one or more selected from the group consisting of a fermented garlic solution fermented by lactic acid bacteria, a concentrate of the same, a dried substance of the same, and a combination thereof.

Another embodiment of the present invention provides a composition for preventing fungi or bacterial infection, which includes a natural antibacterial agent including one or more selected from the group consisting of a fermented garlic solution fermented by lactic acid bacteria, a concentrate of the same, a dried substance of the same, and a combination thereof.

Another embodiment of the present invention provides a method for preventing fungi or microbial infection by using a natural antibacterial substance including one or more selected from the group consisting of a fermented garlic solution made by lactic acid bacteria, a concentrate of the same, a dried substance of the same, and a combination thereof.

Below is provided a more detailed description of the present invention.

For the purpose of the present invention, lactic acid bacteria to be used can be one or more selected from the group consisting of bacteria belonging to genera Weissella, Lactobacillus, Lactococcus, Enterococcus, Streptococcus, and Leuconostoc.

Lactic acid bacteria belonging to genus Weissella can include, but are not limited to, Weissella koreensis, Weissella kimchii, Weissella cibaria, Weissella confusa, and like. For instance, Weissella koreensis KCTC 3621 was used as a representative of the genus of Weissella in an embodiment of the present invention.

Lactic acid bacteria belonging to said genus of Lactobacillus can include, but are not limited to, Lactobacillus fermentum, Lactobacillus acidophilus, Lactobacillus delbrueckii spp. bulgaricus, Lactobacillus plantarum, Lactobacillus casei, Lactobacillus reuteri and like. For instance, Lactobacillus fermentum KCTC 3112, Lactobacillus acidophilus KCTC 3164, and Lactobacillus delbrueckii spp. bulgaricus KCTC 3635, were used as representatives of the genus of Lactobacillus in an embodiment of the present invention.

Lactic acid bacteria belonging to said genus of Lactococcus can include, but are not limited to, Lactococcus lactis spp. lactis, Lactococcus lactis spp. cremoris, and like. For instance, Lactococcus lactis spp. lactis KCTC 3769 was used as a representative of the genus.

Lactic acid bacteria belonging to said genus of Enterococcus can include, but are not limited to, Enterococcus faecium, Enterococcus faecalis, and like. For instance, Enterococcus faecium KCTC 3122 as a representative of the genus of Enterococcus was used in an embodiment of the present invention.

Lactic acid bacteria belonging to said genus of Streptococcus can include, but are not limited to, Streptococcus thermophilus and like. For instance, Streptococcus thermophilus KCTC 3658 as a representative of the genus of Streptococcus was used in an embodiment of the present invention.

Lactic acid bacteria belonging to said genus of Leuconostoc can include, but are not limited to, Leuconostoc lactis, Leuconostoc citreum, Leuconostoc mesenteroides, and like. For instance, Leuconostoc lactis KCTC 3528 as a representative strain of the genus of Leuconostoc was used in an embodiment of the present invention.

Bacteria against which the natural antibacterial agents according to the embodiments of the present invention operate include all types of pathogenic bacteria and fungi, for instance, one or more selected from the group consisting of gram-positive and gram-negative bacteria, yeast, and antibiotic-resistant bacteria.

The gram-positive bacteria can include Staphylococcus aureus, Streptococcus pneumoniae, Streptococcus pyogenes, Enterobacter cloacae, and the like. In an embodiment of the present invention, Staphylococcus aureus KCTC 1621 and Enterobacter cloacae KCTC 2361 may be used as representative examples.

The gram-negative bacteria can include Escherichia coli, Klebsiella pneumoniae, Proteus mirabilis, Haemophilus parainfluenzae, and Pseudomonas aeruginosa. In an embodiment of the present invention, Escherichia coli KCTC 2441 and Klebsiella pneumoniae KCTC 5485 may be used as representative examples.

The yeast can include Candida albicans, Candida glabrata, Candida krusei, and Cryptococcus neoformans. In an embodiment of the present invention, Candida albicans KCTC 7270 may be used as a representative example.

The antibiotic-resistant bacteria can be a strain selected for its antibiotic-resistance to oxytetracycline from fecal excrement of poultry and swine. In an embodiment of the present invention, the strains with strong resistance among the above, such as S008 (Citrobacter werkmanii), PE021 (Citrobacter freund), and PS025 (Escherichia coli), may be used as representative examples.

For the present invention, fermented garlic broth can be obtained by fermenting garlic with lactic acid bacteria, in the form of cell-containing and/or cell-free solution. The concentrate or dried substance of the fermented garlic solution can be obtained through conventional methods for concentrations or drying as known in the art.

For the purpose of the present invention, the fermented garlic solution can be the fermented broth itself or its supernatant collected after centrifuging the fermented broth. The fermented solution is obtainable by adding water to garlic grounds, and then fermenting them with said lactic acid bacteria added. To achieve an appropriate level of pH and the most desired level of antibacterial effect, it is preferred that a mixing ratio (by weight) of garlic grounds and water is within the range of 1:0.5 to 1.2 (garlic ground weight to water weight), with lactic acid bacteria added in the range of 1.0×10⁶ cfu/g to 1.0×10¹² cfu/g (cfu per weight of mixture of garlic grounds and water), more preferably 1.0×10⁸ cfu/g to 1.0×10¹⁰ cfu/g, at the temperatures of 20° C. to 40° C., more preferably 25° C. to 37° C., for 10 to 48 hrs, and more preferably 18 to 30 hrs.

A concentration of the fermented garlic solution as an active ingredient in the antibacterial agent by the present invention can be at least 0.001 vol. %, preferably 1 vol. % or more, and more preferably 3 vol. % or more. The higher the concentration of the active ingredient is, the greater its antibacterial effect will be, although the concentration to be applied can be properly adjusted depending on purposes, application forms, and desired effects of the antibacterial agent, within the range below to the maximum of 100 vol. %. In an embodiment of the present invention, it was observed that antibacterial activity was fully (100%) achieved at concentrations around 5 vol. % or more, and thus, the maximum concentration of the active ingredient in antibacterial compositions may be any value within the range of 5 to 100 vol. %, and more preferably 7 to 100 vol. %. For example, concentrations of the active ingredient in the antibacterial compositions of the present invention may be within the ranges of about 0.1 to 30 vol. %, 1 to 30 vol. %, 1 to 20 vol. %, 3 to 30 vol. %, 3 to 20 vol. %, 3 to 10 vol. %, 5 to 30 vol. %, 5 to 20 vol. %, 5 to 10 vol. %, 7 to 30 vol. %, 7 to 20 vol. %, or 7 to 10 vol. %.

The dried substance of the fermented garlic solution fermented by lactic acid bacteria is obtained with the proportion of about 100 mg per 1 ml of said fermented garlic solution. Therefore, the lower limit of the content of a concentration of the fermented garlic solution, the concentrate of the same, or the dried substance of the same may be at least 0.0001 wt. %, preferably at least 0.1 wt. %, and more preferably at least 0.3 wt. %, and the upper limit may be any value within 0.5 to 100 wt. %, and preferably 0.7 to 100 wt. %, based on dry weight. For example, the content of the active ingredient may be about 0.01 to 3 wt. %, 0.1 to 3 wt. %, 0.1 to 2 wt. %, 0.3 to 3 wt. %, 0.3 to 2 wt. %, 0.3 to 1 wt. %, 0.5 to 3 wt. %, 0.5 to 2 wt. %, 0.5 to 1 wt. %, 0.7 to 3 wt. %, 0.7 to 2 wt. %, or 0.7 to 1 wt. %.

The antibacterial composition according to the present invention can be provided in a form selected from the group consisting of solution, feed, food, medication and cosmetics, depending on the subject to which application will be made and the form of application. Concentrations of the fermented garlic solution fermented by lactic acid bacteria, the concentrate of the same, and/or the dried substance of the same in the antibacterial composition can be adjusted depending on the application form of the composition. The concentrations can be within, but are not limited to, the range of 0.01 to 99.9 vol. % by dry volume, preferably 0.1 to 50 wt. %. The antibacterial composition can father include additives such as excipients, preservatives, and stabilizers, depending on the application form of the composition.

Another aspect of the present invention relates to a method for preventing bacteria, which includes the step of applying the above antibacterial agent and/or composition for preventing bacterial infection to an animal. Animals to which application is made can be any animal, preferably vertebrates such crustaceans, fish, birds, and mammals, and more preferably, shrimps, eels, chickens, ducks, pheasants, pigs, cows, dogs, and humans. Application can be by any conventional administration regimes of oral and parenteral administration (for example rectal, intravenous, intramuscular, and hypodermic administrations). Daily dose can be adjusted according to subject's symptom, age, and condition, and desired effect from the application. For example, by weight of the active ingredient, a daily dosage can be within the range of 0.1 to 500 mg/kg (weight) and preferably 1 to 100 mg/kg (weight) which can be administered at a single dose or multiple doses, although dose amounts and administration regimes are not limited to the example.

As mentioned above, the fermented garlic solution according to the present invention not only exhibits a great antibacterial activity against gram-positive and gram-negative bacteria, yeast, and antibiotic-resistant bacteria, but also possesses lesser toxicity to animal cells than non-fermented garlic does. As such, the fermented garlic solution according to the present invention can be used as a natural antibacterial substance against antibiotic-sensitive bacteria and antibiotic-resistant bacteria.

### EXAMPLES

The present invention is further explained in more detail with reference to the following examples. These examples, however, should not be interpreted as limiting the scope of the present invention in any manner.

### Example 1

### Culture of Lactic Acid Bacteria

Genuses Weissella, Lactobacillus, Lactococcus, Enterococcus, Streptococcus, and Leuconostoc were used to culture lactic acid bacteria. Genus Weissella includes Weissella koreensis, Weissella kimchii, Weissella cibaria, Weissella confuse, and the like, wherein Weissella koreensis KCTC 3621 was used as a representative example. Genus Lactobacillus includes Lactobacillus fermentum, Lactobacillus acidophilus, Lactobacillus delbrueckii spp. bulgaricus, Lactobacillus plantarum, Lactobacillus casei, Lactobacillus reuteri, and the like, wherein Lactobacillus fermentum KCTC 3112, Lactobacillus acidophilus KCTC 3164, and Lactobacillus delbrueckii spp. bulgaricus KCTC 3635 were used as representative examples. Genus Lactococcus includes Lactococcus lactis spp. lactis, Lactococcus lactis spp. cremoris, and the like, wherein Lactococcus lactis spp. lactis KCTC 3769 was used as a representative example. Genus Enterococcus includes Enterococcus faecium, Enterococcus faecalis, and the like, wherein Enterococcus faecium KCTC 3122 was used as a representative example. Genus Streptococcus includes Streptococcus thermophilus, and the like, wherein Streptococcus thermophilus KCTC 3658 was used as a representative example. Genus Leuconostoc includes Leuconostoc lactis, Leuconostoc citreum, Leuconostoc mesenteroides, and the like, wherein Leuconostoc lactis KCTC 3528 was used as a representative example.

2 L of MRS broth (Difco) was subjected to high pressure sterilization at 121° C. for 15 minutes, and then allotted at the amount of 200 mL. Each of Weissella koreensis KCTC 3621, Lactobacillus fermentum KCTC 3112, Lactobacillus acidophilus KCTC 3164, Lactobacillus delbrueckii spp. bulgaricus KCTC 3635, Lactococcus lactis spp. lactis KCTC 3769, Enterococcus faecium KCTC 3122, Streptococcus thermophilus KCTC 3658, and Leuconostoc lactis KCTC 3528 was inoculated in the allotted broth at the concentration of 10⁴ to 10⁶ cfu/ml, and subjected to shaking culture at 25° C. for 24.

### Example 2

### Preparation of Fermented Garlic Solution

Garlic (Allium sativum) and lactic acid bacteria, such as Weissella koreensis KCTC 3621, Lactobacillus fermentum KCTC 3112, Lactobacillus acidophilus KCTC 3164, Lactobacillus delbrueckii spp. bulgaricus KCTC 3635, Lactococcus lactis spp. lactis KCTC 3769, Enterococcus faecium KCTC 3122, Streptococcus thermophilus KCTC 3658, and Leuconostoc lactis KCTC 3528 were used in preparing fermented garlic solutions.

1000 g of ground garlic was added to 1000 g of water, and then allotted in the amount of 200 g. Each of Weissella koreensis KCTC 3621, Lactobacillus fermentum KCTC 3112, Lactobacillus acidophilus KCTC 3164, Lactobacillus delbrueckii spp. bulgaricus KCTC 3635, Lactococcus lactis spp. lactis KCTC 3769, Enterococcus faecium KCTC 3122, Streptococcus thermophilus KCTC 3658, and Leuconostoc lactis KCTC 3528 was added to the allotted grinded garlic solution, to ferment for 24 hours. The obtained fermented solution was centrifuged at 10,000 rpm for 8 minutes, and the supernatant was collected to be used as a fermented garlic solution in the following examples.

The pH and the number of bacteria after fermentation of garlic and centrifugation are as shown in Table 1.

TABLE 1

| | | | Number of |
|---|---|---|---|
| KCTC No. | Name | pH | bacteria (CFU/mL) |
| 3621 | Weissella koreensis | 6.42 | 8.5 × 10⁴ |
| 3112 | Lactobacillus fermentum | 5.90 | 1.5 × 10⁶ |
| 3164 | Lactobacillu acidophilus | 5.79 | 8.3 × 10⁶ |
| 3635 | Lactobacillus delbrueckii spp. | 6.18 | 3.5 × 10⁶ |
| | Bulgaricus | | |
| 3769 | Lactococcus lactis spp. Lactis | 5.91 | 2.4 × 10⁴ |
| 3122 | Enterococcus faecium | 6.22 | 4.5 × 10⁷ |
| 3658 | Streptococcus thermophilus | 6.08 | 3.0 × 10⁶ |
| 3528 | Leuconostoc lactis | 5.98 | 5.0 × 10⁵ |

### Example 3

### Antibacterial Effects of Fermented Garlic Solution Fermented by Lactic Acid Bacteria

The antibacterial effects of fermented garlic solution fermented by lactic acid bacteria were evaluated for gram-positive bacteria, gram-negative bacteria, yeast, and antibiotic-resistant bacteria. The gram-positive bacteria include Staphylococcus aureus, Streptococcus pneumoniae, Streptococcus pyogenes, Enterobacter cloacae, and the like, wherein Staphylococcus aureus KCTC 1621 and Enterobacter cloacae KCTC 2361 were used as representative examples. The gram-negative bacteria include Escherichia coli (KCTC 2441), Klebsiella pneumoniae (KCTC 5485), Proteus mirabilis (KCTC 2566), Haemophilus parainfluenzae, Pseudomonas aeruginosa, and the like, wherein Escherichia coli KCTC 2441 and Klebsiella pneumoniae KCTC 5485 were used as representative examples. Bacteria belonging to yeast include Candida albicans, Candida glabrata, Candida krusei, Cryptococcus neoformans, and the like, wherein Candida albicans KCTC 7270 was used as a representative example. As the antibiotic-resistant bacteria, oxytetracyclin-resistant bacteria were selected from feces of poultry and swine. Out of the selected bacteria, S008 (Citrobacter werkmanii), PE021 (Citrobacter freund), and PS025 (Escherichia coli), which exhibit strong resistance, were used.

The antibacterial effect of the fermented garlic solution was evaluated by a time-kill assay (James H. Jorgensen et al., Antibacterial Agents and Chemotherapy, 1997).

### Example 3-1

### Antibiotic Effect Against Staphylococcus aureus (KCTC 1621)

Staphylococcus aureus KCTC 1621 was diluted with peptone water (16.1 g/L, pH 7.0, Merck) and inoculated in a nutrient broth (Difco) so that the final concentration was 10⁴ to 10⁶ cfu/ml. To the obtained mixture, the cell-containing fermented garlic solutions prepared in Example 2, which are fermented by Weissella koreensis KCTC 3621, Lactobacillus fermentum KCTC 3112, Lactococcus lactis spp. lactis KCTC 3769, Enterococcus faecium KCTC 3122, Streptococcus thermophilus KCTC 3658, and Leuconostoc lactis KCTC 3528, were added at the concentration of 0 vol. % (control), 3 vol. %, 5 vol. %, 7 vol. %, and 10 vol. %, respectively, and the mixtures were cultured at 37° C. The number of living cells of Staphylococcus aureus KCTC 1621 was then measured in nutrient agar (Difco) at 0, 2, 4, 8, and 24 hours, or 0, 4, 8, and 24 hours. The antibacterial effects of the fermented garlic solutions fermented by lactic acid bacteria were compared with those of a non-fermented garlic solution and a culture solution of Weissella koreensis KCTC 3621. The non-fermented garlic solution, which is used as a comparative example, was prepared by mixing 100 g of ground garlic with 100 g of water. The culture solution of Weissella koreensis KCTC 3621, which is used as a comparative example, was prepared by centrifuging the culture solution prepared in Example 1 at 15,000 rpm for 10 minutes, and filtering the supernatant with a 0.2 μm polypropylene syringe filter.

The antibacterial effects of the culture solution of Weissella koreensis, the non-fermented garlic solution, and the fermented garlic solution are shown in Table 2.

TABLE 2
A. Culture solution of Weissella koreensis

Number of living cells
(log₁₀ CFU/mL)

| Sample | Solution | 0 h | 4 h | 8 h | 24 h |
|---|---|---|---|---|---|
| Control | 0 | 5.54 | 6.90 | 8.22 | 8.48 |
| 1 | 3% W. k culture solution | 5.54 | 7.18 | 8.30 | 8.43 |
| 2 | 5% W. k culture solution | 5.54 | 6.95 | 8.19 | 8.18 |
| 3 | 7% W. k culture solution | 5.54 | 7.18 | 8.15 | 8.00 |
| 4 | 10% W. k culture solution | 5.54 | 7.38 | 8.00 | 8.18 |

B. Non-fermented garlic solution

| | | Number of living cells | | | | |
|---|---|---|---|---|---|---|
| | | (log₁₀ CFU/mL) | | | | |
| Sample | Solution | 0 h | 2 h | 4 h | 8 h | 24 h |
| Control | 0 | 5.92 | 6.48 | 9.18 | 9.19 | 8.78 |
| 1 | 3% garlic solution | 5.92 | 6.35 | 8.29 | 8.48 | 8.54 |
| 2 | 5% garlic solution | 5.92 | 6.28 | 7.30 | 7.30 | 5.00 |
| 3 | 7% garlic solution | 5.92 | 5.88 | 6.70 | 6.40 | 4.48 |
| 4 | 10% garlic solution | 5.92 | 5.93 | 6.00 | 5.18 | 0.00 |

| | | Number of living cells | | | | |
|---|---|---|---|---|---|---|
| | | (log₁₀ CFU/mL) | | | | |
| Sample | Solution | 0 | 2 | 4 | 8 | 24 |

C. Fermented garlic solution (Weissella koreensis)

| | | | | | | |
|---|---|---|---|---|---|---|
| Control | 0 | 3.78 | 4.41 | 5.06 | 7.04 | 9.46 |
| 1 | 3% W. k fermented garlic solution | 3.78 | 4.11 | 4.48 | 4.54 | 7.18 |
| 2 | 5% W. k fermented garlic solution | 3.78 | 3.93 | 4.00 | 3.70 | 3.54 |
| 3 | 7% W. k fermented garlic solution | 3.78 | 3.74 | 3.65 | 0.00 | 0.00 |
| 4 | 10% W. k fermented garlic solution | 3.78 | 3.74 | 3.40 | 0.00 | 0.00 |

D. Fermented garlic solution (Lactobacillus fermentum)

| | | | | | | |
|---|---|---|---|---|---|---|
| Control | 0 | 3.78 | 4.41 | 5.06 | 7.04 | 9.46 |
| 1 | 3% Lb. fermentum fermented garlic solution | 3.78 | 4.11 | 4.74 | 5.18 | 6.81 |
| 2 | 5% Lb. fermentum fermented garlic solution | 3.78 | 3.88 | 4.18 | 4.70 | 6.60 |
| 3 | 7% Lb. fermentum fermented garlic solution | 3.78 | 3.85 | 4.48 | 4.40 | 3.70 |
| 4 | 10% Lb. fermentum fermented garlic solution | 3.78 | 3.65 | 3.48 | 0.00 | 0.00 |

E. Fermented garlic solution (Lactococcus lactis spp. lactis)

| | | | | | | |
|---|---|---|---|---|---|---|
| Control | 0 | 5.31 | 6.54 | 7.60 | 8.40 | 8.74 |
| 1 | 3% Lactococcus lactis fermented garlic solution | 5.31 | 5.54 | 6.03 | 5.90 | 0.00 |
| 2 | 5% Lactococcus lactis fermented garlic solution | 5.31 | 5.81 | 5.54 | 5.24 | 0.00 |
| 3 | 7% Lactococcus lactis fermented garlic solution | 5.31 | 5.60 | 5.30 | 5.00 | 0.00 |
| 4 | 10% Lactococcus lactis fermented garlic solution | 5.31 | 5.54 | 5.40 | 4.81 | 0.00 |

F. Fermented garlic solution (Enterococcus faecium)

| | | Number of living cells | | | |
|---|---|---|---|---|---|
| | | (log₁₀ CFU/mL) | | | |
| Sample | Solution | 0 | 4 | 8 | 24 |
| Control | 0 | 5.81 | 7.23 | 8.30 | 8.90 |
| 1 | 3% Enterococcus faecium fermented garlic solution | 5.81 | 7.04 | 7.70 | 8.18 |
| 2 | 5% Enterococcus faecium fermented garlic solution | 5.81 | 6.48 | 5.70 | 5.44 |
| 3 | 7% Enterococcus faecium fermented garlic solution | 5.81 | 5.78 | 4.70 | 0.00 |
| 4 | 10% Enterococcus faecium fermented garlic solution | 5.81 | 5.85 | 5.65 | 0.00 |

G. Fermented garlic solution (Streptococcus thermophilus)

| | | Number of living cells | | | | |
|---|---|---|---|---|---|---|
| | | (log₁₀ CFU/mL) | | | | |
| Sample | Solution | 0 | 2 | 4 | 8 | 24 |
| Control | 0 | 5.65 | 6.03 | 7.04 | 8.48 | 8.70 |
| 1 | 3% Streptococcus thermophilus fermented garlic solution | 5.65 | 6.06 | 6.22 | 6.18 | 7.40 |
| 2 | 5% Streptococcus thermophilus fermented garlic solution | 5.65 | 5.54 | 5.55 | 4.92 | 3.60 |
| 3 | 7% Streptococcus thermophilus fermented garlic solution | 5.65 | 6.00 | 5.01 | 4.57 | 2.70 |
| 4 | 10% Streptococcus thermophilus fermented garlic | 5.65 | 5.48 | 4.68 | 4.24 | 0.00 |
| | solution | | | | | |

H. Fermented garlic solution (Leuconostoc lactis)

| | | Number of living cells | | | |
|---|---|---|---|---|---|
| | | (log₁₀ CFU/mL) | | | |
| Sample | Solution | 0 | 4 | 8 | 24 |
| Control | 0 | 5.81 | 7.23 | 8.30 | 8.90 |
| 1 | 3% Leuconostoc lactis fermented garlic solution | 5.81 | 6.40 | 7.02 | 6.65 |
| 2 | 5% Leuconostoc lactis fermented garlic solution | 5.81 | 6.60 | 5.18 | 0.00 |
| 3 | 7% Leuconostoc lactis fermented garlic solution | 5.81 | 6.00 | 4.18 | 0.00 |
| 4 | 10% Leuconostoc lactis fermented garlic solution | 5.81 | 5.81 | 3.74 | 0.00 |

As shown in Table 2, the culture solution of Weissella koreensis shows no antibacterial effect against Staphylococcus aureus KCTC 1621 even at the concentration of 10 vol. %, and thus no meaningful effect of cell death is observed. The non-fermented garlic solution exhibits antibacterial effect only at the concentration of 10 vol. %. However, the fermented garlic solution fermented by lactic acid bacteria according to an embodiment of the present invention exhibits a considerably superior antibacterial effect compared with the culture solution of Weissella koreensis and the non-fermented garlic solution, and in particular, shows a complete antibacterial effect of 100% at the concentration of 3 vol. % or more, and more surely at 10 vol. % or more. From such results of Table 2, it is confirmed that the fermented garlic solution fermented by lactic acid bacteria according to an embodiment of the present invention exhibits considerably superior antibacterial effects than those of the culture solution of lactic acid bacteria and non-fermented garlic solution.

### Example 3-2

### Antibacterial Effect Against Enterobacter cloacae (KCTC 2361)

Enterobacter cloacae KCTC 2361 was diluted with peptone water (16.1 g/L, pH 7.0, Merck) and inoculated in a nutrient broth (Difco) so that the final concentration was 10⁴ to 10⁶ cfu/ml. To the obtained mixture, the cell-containing fermented garlic solutions prepared in Example 2, which are fermented by Weissella koreensis KCTC 3621, Lactobacillus fermentum KCTC 3112, Lactobacillus acidophilus KCTC 3164, Lactobacillus delbrueckii spp. bulgaricus KCTC 3635, Lactococcus lactis spp. lactis KCTC 3769, Enterococcus faecium KCTC 3122, Streptococcus thermophilus KCTC 3658, and Leuconostoc lactis KCTC 3528, were added at the concentration of 0 vol. % (control), 3 vol. %, 5 vol. %, 7 vol. %, and 10 vol. %, respectively, and the mixtures were cultured at 37° C. The number of living cells of Enterobacter cloacae KCTC 2361 was then measured in nutrient agar (Difco) at 0, 2, 4, 8, and 24 hours, or 0, 4, 8, and 24 hours.

The antibacterial effects of the fermented garlic solutions fermented by lactic acid bacteria were compared with those of a non-fermented garlic solution and a culture solution of Weissella koreensis KCTC 3621. The non-fermented garlic solution, which is used as a comparative example, was prepared by mixing 100 g of ground garlic with 100 g of water. The culture solution of Weissella koreensis KCTC 3621, which is used as a comparative example, was prepared by centrifuging the culture solution prepared in Example 1 at 15,000 rpm for 10 minutes, and filtering the supernatant with a 0.2 μm polypropylene syringe filter.

The antibacterial effects of the culture solution of Weissella koreensis, the non-fermented garlic solution, and the fermented garlic solution are shown in Table 3.

TABLE 3

Number of living
cells (log₁₀ CFU/mL)

| | | | | | |
|---|---|---|---|---|---|
| Sample | Solution | 0 h | 4 h | 8 h | 24 h |

A. Culture solution of Weissella koreensis

| | | | | | |
|---|---|---|---|---|---|
| Control | 0 | 5.78 | 8.48 | 8.85 | 9.08 |
| 1 | 3% W. k culture solution | 5.78 | 8.90 | 8.65 | 9.00 |
| 2 | 5% W. k culture solution | 5.78 | 8.18 | 8.40 | 8.81 |
| 3 | 7% W. k culture solution | 5.78 | 7.70 | 8.38 | 9.15 |
| 4 | 10% W. k culture solution | 5.78 | 7.48 | 7.95 | 8.70 |

B. Non-fermented garlic solution

| | | | | | |
|---|---|---|---|---|---|
| Control | 0 | 6.60 | 8.00 | 9.06 | 9.11 |
| 1 | 3% garlic solution | 6.60 | 8.54 | 9.48 | 9.19 |
| 2 | 5% garlic solution | 6.60 | 8.40 | 9.24 | 9.18 |
| 3 | 7% garlic solution | 6.60 | 8.10 | 9.27 | 9.18 |
| 4 | 10% garlic solution | 6.60 | 6.70 | 8.78 | 9.26 |

Number of living
cells (log₁₀ CFU/mL)

| | | | | | |
|---|---|---|---|---|---|
| Sample | Solution | 0 | 4 | 8 | 24 |

C. Fermented garlic solution (Weissella koreensis)

| | | | | | |
|---|---|---|---|---|---|
| Control | 0 | 6.70 | 8.95 | 9.33 | 9.42 |
| 1 | 3% W. k fermented garlic solution | 6.70 | 7.70 | 9.28 | 9.57 |
| 2 | 5% W. k fermented garlic solution | 6.70 | 6.74 | 6.00 | 9.56 |
| 3 | 7% W. k fermented garlic solution | 6.70 | 5.95 | 5.81 | 0.00 |
| 4 | 10% W. k fermented garlic solution | 6.70 | 6.10 | 5.00 | 0.00 |

D. Fermented garlic solution (Lactobacillus fermentum)

| | | | | | |
|---|---|---|---|---|---|
| Control | 0 | 6.70 | 8.95 | 9.33 | 9.42 |
| 1 | 3% Lb. fermentum fermented garlic solution | 6.70 | 9.37 | 9.10 | 9.48 |
| 2 | 5% Lb. fermentum fermented garlic solution | 6.70 | 8.90 | 8.06 | 9.42 |
| 3 | 7% Lb. fermentum fermented garlic solution | 6.70 | 7.54 | 5.31 | 5.18 |
| 4 | 10% Lb. fermentum fermented garlic solution | 6.70 | 6.30 | 4.98 | 5.40 |

E. Fermented garlic solution (Lactobacillus acidophilus)

| | | | | | |
|---|---|---|---|---|---|
| Control | 0 | 6.70 | 8.95 | 9.33 | 9.42 |
| 1 | 3% Lb. acidophilus fermented garlic solution | 6.70 | 8.28 | 9.08 | 9.50 |
| 2 | 5% Lb. acidophilus fermented garlic solution | 6.70 | 7.30 | 6.88 | 9.35 |
| 3 | 7% Lb. acidophilus fermented garlic solution | 6.70 | 6.54 | 5.60 | 7.56 |
| 4 | 10% Lb. acidophilus fermented garlic solution | 6.70 | 6.06 | 4.54 | 0.00 |

F. Fermented garlic solution (Lactobacillus delbrueckii spp. bulgaricus)

| | | | | | |
|---|---|---|---|---|---|
| Control | 0 | 6.70 | 8.95 | 9.33 | 9.42 |
| 1 | 3% Lb. Bulgaricus fermented garlic solution | 6.70 | 9.33 | 8.88 | 9.45 |
| 2 | 5% Lb. Bulgaricus fermented garlic solution | 6.70 | 8.30 | 6.78 | 9.39 |
| 3 | 7% Lb. Bulgaricus fermented garlic solution | 6.70 | 6.60 | 5.30 | 6.95 |
| 4 | 10% Lb. Bulgaricus fermented garlic solution | 6.70 | 6.18 | 4.88 | 0.00 |

G. Fermented garlic solution (Lactococcus lactis spp. lactis)

| | | | | | |
|---|---|---|---|---|---|
| Control | 0 | 6.70 | 8.95 | 9.33 | 9.42 |
| 1 | 3% Lactococcus lactis fermented garlic solution | 6.70 | 6.65 | 9.08 | 9.64 |
| 2 | 5% Lactococcus lactis fermented garlic solution | 6.70 | 6.48 | 7.08 | 6.85 |
| 3 | 7% Lactococcus lactis fermented garlic solution | 6.70 | 6.00 | 4.18 | 0.00 |
| 4 | 10% Lactococcus lactis fermented garlic solution | 6.70 | 6.24 | 4.65 | 0.00 |

| | | Number of living | | | | |
|---|---|---|---|---|---|---|
| | | cells (log₁₀ CFU/mL) | | | | |
| Sample | Solution | 0 | 2 | 4 | 8 | 24 |

H. Fermented garlic solution (Enterococcus faecium)

| | | | | | | |
|---|---|---|---|---|---|---|
| Control | 0 | 5.97 | 7.24 | 7.95 | 8.37 | 9.22 |
| 1 | 3% Enterococcus faecium fermented garlic solution | 5.97 | 6.70 | 7.10 | 8.30 | 8.98 |
| 2 | 5% Enterococcus faecium fermented garlic solution | 5.97 | 6.00 | 5.95 | 6.06 | 8.93 |
| 3 | 7% Enterococcus faecium fermented garlic solution | 5.97 | 5.70 | 6.18 | 6.04 | 5.18 |
| 4 | 10% Enterococcus faecium fermented garlic solution | 5.97 | 5.93 | 5.88 | 4.70 | 0.00 |

I. Fermented garlic solution (Streptococcus thermophilus)

| | | | | | | |
|---|---|---|---|---|---|---|
| Control | 0 | 5.13 | 7.20 | 8.15 | 8.54 | 9.18 |
| 1 | 3% Streptococcus thermophilus fermented garlic solution | 5.13 | 5.88 | 7.25 | 5.00 | 9.32 |
| 2 | 5% Streptococcus thermophilus fermented garlic solution | 5.13 | 5.42 | 6.51 | 4.40 | 8.48 |
| 3 | 7% Streptococcus thermophilus fermented garlic solution | 5.13 | 5.03 | 4.83 | 4.00 | 0.00 |
| 4 | 10% Streptococcus thermophilus fermented garlic solution | 5.13 | 5.06 | 4.74 | 3.00 | 0.00 |

J. Fermented garlic solution (Leuconostoc lactis)

| | | Number of living | | | |
|---|---|---|---|---|---|
| | | cells (log₁₀ CFU/mL) | | | |
| Sample | Solution | 0 | 4 | 8 | 24 |
| Control | 0 | 6.70 | 8.95 | 9.33 | 9.42 |
| 1 | 3% Leuconostoc lactis fermented garlic solution | 6.70 | 8.54 | 7.04 | 9.53 |
| 2 | 5% Leuconostoc lactis fermented garlic solution | 6.70 | 5.90 | 4.18 | 0.00 |
| 3 | 7% Leuconostoc lactis fermented garlic solution | 6.70 | 5.48 | 4.00 | 0.00 |
| 4 | 10% Leuconostoc lactis fermented garlic solution | 6.70 | 5.78 | 3.00 | 0.00 |

As shown in Table 3, the culture solution of Weissella koreensis and the non-fermented garlic solution show no antibacterial effect against Enterobacter cloacae KCTC 2361 even at the concentration of 10 vol. %, and thus no meaningful effect of cell death is observed. However, the fermented garlic solution fermented by lactic acid bacteria according to an embodiment of the present invention exhibits a considerably superior antibacterial effect compared with the culture solution of Weissella koreensis and the non-fermented garlic solution, and in particular, shows a complete antibacterial effect of 100% at the concentration of 5 vol. % or more, and more surely at 10 vol. % or more. From such results of Table 3, it is confirmed that the fermented garlic solution fermented by lactic acid bacteria according to an embodiment of the present invention exhibits considerably superior antibacterial effects than those of the fermented solution of lactic acid bacteria and non-fermented garlic solution.

### Example 3-3

### Antibacterial effect against Escherichia coli (KCTC 2441)

Escherichia coli KCTC 2441 was diluted with peptone water (16.1 g/L, pH 7.0, Merck) and inoculated in a nutrient broth (Difco) so that the final concentration was 10⁴ to 10⁶ cfu/ml. To the obtained mixture, the cell-containing fermented garlic solutions prepared in Example 2, which are fermented by Weissella koreensis KCTC 3621, Lactococcus lactis spp. lactis KCTC 3769, Enterococcus faecium KCTC 3122, Streptococcus thermophilus KCTC 3658, and Leuconostoc lactis KCTC 3528, were added at the concentration of 0 vol. % (control), 3 vol. %, 5 vol. %, 7 vol. %, and 10 vol. %, respectively, and the mixtures were cultured at 37° C. The number of living cells of Escherichia coli KCTC 2441 was then measured in nutrient agar at 0, 2, 4, 8, and 24 hours, or 0, 4, 8, and 24 hours.

The antibacterial effects of the fermented garlic solutions fermented by lactic acid bacteria were compared with those of a non-fermented garlic solution and a culture solution of Weissella koreensis KCTC 3621. The non-fermented garlic solution, which is used as a comparative example, was prepared by mixing 100 g of ground garlic with 100 g of water. The culture solution of Weissella koreensis KCTC 3621, which is used as a comparative example, was prepared by centrifuging the culture solution prepared in Example 1 at 15,000 rpm for 10 minutes, and filtering the supernatant with a 0.2 μm polypropylene syringe filter.

The antibacterial effects of the culture solution of Weissella koreensis, the non-fermented garlic solution, and the fermented garlic solution are shown in Table 4.

TABLE 4

Number of living
cells (log₁₀ CFU/mL)

| | | | | | |
|---|---|---|---|---|---|
| Sample | Solution | 0 | 4 | 8 | 24 |

A. Culture solution of Weissella koreensis

| | | | | | |
|---|---|---|---|---|---|
| Control | 0 | 6.00 | 9.04 | 8.65 | 9.11 |
| 1 | 3% W. k culture solution | 6.00 | 8.78 | 9.00 | 9.11 |
| 2 | 5% W. k culture solution | 6.00 | 8.48 | 8.88 | 8.60 |
| 3 | 7% W. k culture solution | 6.00 | 7.88 | 8.74 | 8.78 |
| 4 | 10% W. k culture solution | 6.00 | 8.00 | 8.90 | 8.40 |

B. Non-fermented garlic solution

| | | | | | |
|---|---|---|---|---|---|
| Control | 0 | 6.60 | 8.19 | 9.24 | 9.02 |
| 1 | 3% garlic solution | 6.60 | 8.00 | 9.34 | 9.20 |
| 2 | 5% garlic solution | 6.60 | 8.60 | 9.37 | 8.95 |
| 3 | 7% garlic solution | 6.60 | 7.81 | 9.28 | 9.18 |
| 4 | 10% garlic solution | 6.60 | 6.04 | 4.70 | 0.00 |

C. Fermented garlic solution (Weissella koreensis)

| | | Number of living | | | | |
|---|---|---|---|---|---|---|
| | | cells (log₁₀ CFU/mL) | | | | |
| Sample | Solution | 0 | 2 | 4 | 8 | 24 |
| Control | 0 | 4.68 | 4.90 | 7.98 | 8.54 | 9.10 |
| 1 | 3% W. k fermented garlic solution | 4.68 | 4.90 | 5.30 | 5.99 | 8.78 |
| 2 | 5% W. k fermented garlic solution | 4.68 | 4.90 | 4.70 | 3.58 | 7.89 |
| 3 | 7% W. k fermented garlic solution | 4.68 | 4.40 | 4.60 | 3.00 | 0.00 |
| 4 | 10% W. k fermented garlic solution | 4.68 | 4.52 | 4.48 | 0.00 | 0.00 |

D. Fermented garlic solution (Lactococcus lactis spp. lactis)

Number of living
cells (log₁₀ CFU/mL)

| Sample | Solution | 0 | 4 | 8 | 24 |
|---|---|---|---|---|---|
| Control | 0 | 9.16 | 8.33 | 9.00 | 9.13 |
| 1 | 3% Lactococcus lactis fermented garlic solution | 9.16 | 7.88 | 7.40 | 8.70 |
| 2 | 5% Lactococcus lactis fermented garlic solution | 9.16 | 8.06 | 6.30 | 0.00 |
| 3 | 7% Lactococcus lactis fermented garlic solution | 9.16 | 5.18 | 2.70 | 0.00 |
| 4 | 10% Lactococcus lactis fermented garlic solution | 9.16 | 3.60 | 0.00 | 0.00 |

| | | Number of living | | | | |
|---|---|---|---|---|---|---|
| | | cells (log₁₀ CFU/mL) | | | | |
| Sample | Solution | 0 | 2 | 4 | 8 | 24 |

E. Fermented garlic solution (Enterococcus faecium)

| | | | | | | |
|---|---|---|---|---|---|---|
| Control | 0 | 5.18 | 6.40 | 7.70 | 8.37 | 8.81 |
| 1 | 3% Enterococcus faecium fermented garlic solution | 5.18 | 5.18 | 5.30 | 5.65 | 8.48 |
| 2 | 5% Enterococcus faecium fermented garlic solution | 5.18 | 5.11 | 5.70 | 4.85 | 0.00 |
| 3 | 7% Enterococcus faecium fermented garlic solution | 5.18 | 5.53 | 5.54 | 4.65 | 0.00 |
| 4 | 10% Enterococcus faecium fermented garlic solution | 5.18 | 5.67 | 4.94 | 3.70 | 0.00 |

F. Fermented garlic solution (Streptococcus thermophilus)

| | | | | | | |
|---|---|---|---|---|---|---|
| Control | 0 | 5.45 | 7.11 | 8.65 | 9.15 | 9.31 |
| 1 | 3% Streptococcus thermophilus fermented garlic solution | 5.45 | 5.60 | 5.45 | 8.60 | 8.44 |
| 2 | 5% Streptococcus thermophilus fermented garlic solution | 5.45 | 4.88 | 5.03 | 6.71 | 0.00 |
| 3 | 7% Streptococcus thermophilus fermented garlic solution | 5.45 | 4.88 | 4.60 | 3.70 | 0.00 |
| 4 | 10% Streptococcus thermophilus fermented garlic solution | 5.45 | 4.48 | 4.00 | 3.30 | 0.00 |

G. Fermented garlic solution (Leuconostoc lactis)

Number of living
cells (log₁₀ CFU/mL

| Sample | Solution | 0 | 4 | 8 | 24 |
|---|---|---|---|---|---|
| Control | 0 | 9.16 | 8.33 | 9.00 | 9.13 |
| 1 | 3% Leuconostoc lactis fermented garlic solution | 9.16 | 7.88 | 6.70 | 8.70 |
| 2 | 5% Leuconostoc lactis fermented garlic solution | 9.16 | 6.22 | 5.00 | 2.70 |
| 3 | 7% Leuconostoc lactis fermented garlic solution | 9.16 | 4.48 | 3.70 | 0.00 |
| 4 | 10% Leuconostoc lactis fermented garlic solution | 9.16 | 2.70 | 0.00 | 0.00 |

As shown in Table 4, the culture solution of Weissella koreensis shows a very low antibacterial effect against Escherichia coli KCTC 2441 even at the concentration of 10 vol. %, and thus no meaningful effect of cell death is observed. The non-fermented garlic solution exhibits antibacterial effect only at the concentration of 10 vol. %. However, the fermented garlic solution fermented by lactic acid bacteria according to an embodiment of the present invention exhibits considerably superior antibacterial effect compared with the culture solution of Weissella koreensis and the non-fermented garlic solution, and in particular, shows a complete antibacterial effect of 100% at the concentration of 5 vol. % or more, and more surely at 10 vol. % or more. From such results of Table 4, it is confirmed that the fermented garlic solution fermented by lactic acid bacteria according to an embodiment of the present invention exhibits considerably superior antibacterial effects than those of the culture solution of lactic acid bacteria and non-fermented garlic solution.

### Example 3-4

### Antibacterial Effect Against Klebsiella pneumoniae (KCTC 5485)

Klebsiella pneumoniae KCTC 5485 was diluted with peptone water (16.1 g/L, pH 7.0, Merck) and inoculated in a nutrient broth (Difco) so that the final concentration was 10⁴ to 10⁶ cfu/ml. To the obtained mixture, the cell-containing fermented garlic solutions prepared in Example 2, which are fermented by Weissella koreensis KCTC 3621, Lactobacillus delbrueckii spp. bulgaricus KCTC 3635, Lactococcus lactis spp. lactis KCTC 3769, and Leuconostoc lactis KCTC 3528, were added at the concentration of 0 vol. % (control), 3 vol. %, 5 vol. %, 7 vol. %, and 10 vol. %, respectively, and the mixtures were cultured at 37° C. The number of living cells of Klebsiella pneumoniae KCTC 5485 was then measured in nutrient agar at 0, 2, 4, 8, and 24 hours, or 0, 4, 8, and 24 hours.

The antibacterial effects of the fermented garlic solutions fermented by lactic acid bacteria were compared with those of a non-fermented garlic solution and a culture solution of Weissella koreensis KCTC 3621. The non-fermented garlic solution, which is used as a comparative example, was prepared by mixing 100 g of ground garlic with 100 g of water. The culture solution of Weissella koreensis KCTC 3621, which is used as a comparative example, was prepared by centrifuging the culture solution prepared in Example 1 at 15,000 rpm for 10 minutes, and filtering the supernatant with 0.2 μm polypropylene syringe filter.

The antibacterial effects of the culture solution of Weissella koreensis, the non-fermented garlic solution, and the fermented garlic solution are shown in Table 5.

TABLE 5

| | | Number of living | | | |
|---|---|---|---|---|---|
| | | cells (log₁₀ CFU/mL) | | | |
| Sample | Solution | 0 | 4 | 8 | 24 |

A. Culture solution of Weissella koreensis

| | | | | | |
|---|---|---|---|---|---|
| Control | 0 | 5.85 | 8.70 | 8.48 | 8.54 |
| 1 | 3% W. k culture solution | 5.85 | 8.88 | 8.98 | 9.22 |
| 2 | 5% W. k culture solution | 5.85 | 8.40 | 8.93 | 8.81 |
| 3 | 7% W. k culture solution | 5.85 | 8.00 | 8.48 | 8.85 |
| 4 | 10% W. k culture solution | 5.85 | 7.54 | 7.95 | 8.65 |

B. Non-fermented garlic solution

| | | | | | |
|---|---|---|---|---|---|
| Control | 0 | 6.54 | 8.18 | 8.88 | 8.48 |
| 1 | 3% garlic solution | 6.54 | 8.88 | 9.16 | 8.70 |
| 2 | 5% garlic solution | 6.54 | 8.60 | 9.13 | 8.98 |
| 3 | 7% garlic solution | 6.54 | 8.65 | 9.00 | 9.00 |
| 4 | 10% garlic solution | 6.54 | 6.70 | 6.30 | 0.00 |

C. Fermented garlic solution (Weissella koreensis)

| | | Number of living | | | | |
|---|---|---|---|---|---|---|
| | | cells (log₁₀ CFU/mL) | | | | |
| Sample | Solution | 0 | 2 | 4 | 8 | 24 |
| Control | 0 | 5.92 | 6.60 | 8.10 | 8.20 | 9.00 |
| 1 | 3% W. k fermented garlic solution | 5.92 | 6.26 | 7.11 | 7.74 | 6.00 |
| 2 | 5% W. k fermented garlic solution | 5.92 | 5.54 | 6.52 | 6.52 | 8.74 |
| 3 | 7% W. k fermented garlic solution | 5.92 | 5.90 | 4.63 | 3.18 | 0.00 |
| 4 | 10% W. k fermented garlic solution | 5.92 | 5.20 | 4.54 | 0.00 | 0.00 |

| | | Number of living | | | |
|---|---|---|---|---|---|
| | | cells (log₁₀ CFU/mL) | | | |
| Sample | Solution | 0 | 4 | 8 | 24 |

D. Fermented garlic solution (Lactobacillus delbrueckii spp. bulgaricus)

| | | | | | |
|---|---|---|---|---|---|
| Control | 0 | 5.40 | 8.78 | 8.18 | 8.40 |
| 1 | 3% Lb. Bulgaricus fermented garlic solution | 5.40 | 8.48 | 8.60 | 7.18 |
| 2 | 5% Lb. Bulgaricus fermented garlic solution | 5.40 | 6.30 | 5.70 | 8.00 |
| 3 | 7% Lb. Bulgaricus fermented garlic solution | 5.40 | 7.30 | 7.18 | 6.00 |
| 4 | 10% Lb. Bulgaricus fermented garlic solution | 5.40 | 5.74 | 4.88 | 0.00 |

E. Fermented garlic solution (Lactococcus lactis spp. lactis)

| | | | | | |
|---|---|---|---|---|---|
| Control | 0 | 5.40 | 8.78 | 8.18 | 8.40 |
| 1 | 3% Lactococcus lactis fermented garlic solution | 5.40 | 7.00 | 6.70 | 5.00 |
| 2 | 5% Lactococcus lactis fermented garlic solution | 5.40 | 6.00 | 5.70 | 5.26 |
| 3 | 7% Lactococcus lactis fermented garlic solution | 5.40 | 4.40 | 0.00 | 0.00 |
| 4 | 10% Lactococcus lactis fermented garlic solution | 5.40 | 4.65 | 0.00 | 0.00 |

F. Fermented garlic solution (Leuconostoc lactis)

| | | | | | |
|---|---|---|---|---|---|
| Control | 0 | 5.40 | 8.78 | 8.18 | 8.40 |
| 1 | 3% Leuconostoc lactis fermented garlic solution | 5.40 | 8.54 | 8.00 | 4.00 |
| 2 | 5% Leuconostoc lactis fermented garlic solution | 5.40 | 5.74 | 4.18 | 0.00 |
| 3 | 7% Leuconostoc lactis fermented garlic solution | 5.40 | 5.30 | 4.06 | 0.00 |
| 4 | 10% Leuconostoc lactis fermented garlic solution | 5.40 | 6.30 | 5.18 | 0.00 |

As shown in Table 5, the culture solution of Weissella koreensis shows a very low antibacterial effect against Klebsiella pneumoniae KCTC 5485 even at the concentration of 10 vol. %, and thus no meaningful effect of cell death is observed. The non-fermented garlic solution exhibits antibacterial effect only at the concentration of 10 vol. %. However, the fermented garlic solution fermented by lactic acid bacteria according to an embodiment of the present invention exhibits considerably superior antibacterial effect compared with the culture solution of Weissella koreensis and the non-fermented garlic solution, and in particular, shows a complete antibacterial effect of 100% at the concentration of 5 vol. % or more, and more surely at 10 vol. % or more. From such results of Table 5, it is confirmed that the fermented garlic solution fermented by lactic acid bacteria according to an embodiment of the present invention exhibits considerably superior antibacterial effects than those of the culture solution of lactic acid bacteria and non-fermented garlic solution.

### Example 3-5

### Antibacterial Effect Against Candida albicans (KCTC 7270)

Candida albicans KCTC 7270 was diluted with peptone water (16.1 g/L, pH 7.0, Merck) and inoculated in a nutrient broth (Difco) so that the final concentration was 10⁴ to 10⁶ cfu/ml. To the obtained mixture, the cell-containing fermented garlic solutions prepared in Example 2, which are fermented by Weissella koreensis KCTC 3621, Lactobacillus fermentum KCTC 3112, Lactococcus lactis spp. lactis KCTC 3769, Streptococcus thermophilus KCTC 3658, and Leuconostoc lactis KCTC 3528, were added at the concentration of 0 vol. % (control), 3 vol. %, 5 vol. %, 7 vol. %, and 10 vol. %, respectively, and the mixtures were cultured at 37° C. The number of living cells of Candida albicans KCTC 7270 was then measured in nutrient agar at 0, 2, 4, 8, and 24 hours, or 0, 4, 8, and 24 hours.

The antibacterial effects of the fermented garlic solutions fermented by lactic acid bacteria were compared with those of a non-fermented garlic solution and a culture solution of Weissella koreensis KCTC 3621. The non-fermented garlic solution, which is used as a comparative example, was prepared by mixing 100 g of ground garlic with 100 g of water. The culture solution of Weissella koreensis KCTC 3621, which is used as a comparative example, was prepared by centrifuging the culture solution prepared in Example 1 at 15,000 rpm for 10 minutes, and filtering the supernatant with a 0.2 μm polypropylene syringe filter.

The antibacterial effects of the culture solution of Weissella koreensis, the non-fermented garlic solution, and the fermented garlic solution are shown in Table 6.

TABLE 6

| | | Number of living | | | |
|---|---|---|---|---|---|
| | | cells (log₁₀ CFU/mL) | | | |
| Sample | Solution | 0 | 4 | 8 | 24 |

A. Culture solution of Weissella koreensis

| | | | | | |
|---|---|---|---|---|---|
| Control | 0 | 6.98 | 9.10 | 8.60 | 9.02 |
| 1 | 3% W. k culture solution | 6.98 | 9.32 | 8.81 | 9.11 |
| 2 | 5% W. k culture solution | 6.98 | 9.24 | 9.00 | 9.08 |
| 3 | 7% W. k culture solution | 6.98 | 9.00 | 8.88 | 8.70 |
| 4 | 10% W. k culture solution | 6.98 | 9.08 | 8.65 | 8.48 |

B. Non-fermented garlic solution

| | | | | | |
|---|---|---|---|---|---|
| Control | 0 | 5.00 | 8.10 | 9.00 | 8.93 |
| 1 | 3% garlic solution | 5.00 | 6.30 | 7.60 | 8.85 |
| 2 | 5% garlic solution | 5.00 | 6.00 | 7.54 | 8.74 |
| 3 | 7% garlic solution | 5.00 | 7.40 | 9.22 | 8.98 |
| 4 | 10% garlic solution | 5.00 | 0.00 | 0.00 | 0.00 |

C. Fermented garlic solution (Weissella koreensis)

| | | | | | |
|---|---|---|---|---|---|
| Control | 0 | 5.76 | 8.11 | 8.74 | 9.11 |
| 1 | 3% W. k fermented garlic solution | 5.76 | 6.46 | 8.88 | 9.10 |
| 2 | 5% W. k fermented garlic solution | 5.76 | 5.70 | 5.18 | 4.60 |
| 3 | 7% W. k fermented garlic solution | 5.76 | 5.11 | 3.00 | 0.00 |
| 4 | 10% W. k fermented garlic solution | 5.76 | 4.48 | 0.00 | 0.00 |

D. Fermented garlic solution (Lactobacillus fermentum)

| | | | | | |
|---|---|---|---|---|---|
| Control | 0 | 4.74 | 9.10 | 9.15 | 8.81 |
| 1 | 3% Lb. fermentum fermented garlic solution | 4.74 | 6.85 | 7.00 | 8.81 |
| 2 | 5% Lb. fermentum fermented garlic solution | 4.74 | 6.00 | 5.18 | 9.04 |
| 3 | 7% Lb. fermentum fermented garlic solution | 4.74 | 4.74 | 3.65 | 8.18 |
| 4 | 10% Lb. fermentum fermented garlic solution | 4.74 | 4.00 | 3.00 | 0.00 |

E. Fermented garlic solution (Lactococcus lactis spp. lactis)

| | | | | | |
|---|---|---|---|---|---|
| Control | 0 | 4.74 | 9.10 | 9.15 | 8.81 |
| 1 | 3% Lactococcus lactis fermented garlic solution | 4.74 | 6.20 | 5.85 | 4.70 |
| 2 | 5% Lactococcus lactis fermented garlic solution | 4.74 | 6.16 | 5.40 | 0.00 |
| 3 | 7% Lactococcus lactis fermented garlic solution | 4.74 | 5.60 | 4.48 | 0.00 |
| 4 | 10% Lactococcus lactis fermented garlic solution | 4.74 | 0.00 | 0.00 | 0.00 |

F. Fermented garlic solution (Streptococcus thermophilus)

| | | | | | |
|---|---|---|---|---|---|
| Control | 0 | 4.74 | 9.10 | 9.15 | 8.81 |
| 1 | 3% Streptococcus thermophilus fermented | 4.74 | 7.90 | 8.60 | 8.90 |
| | garlic solution | | | | |
| 2 | 5% Streptococcus thermophilus fermented | 4.74 | 7.81 | 8.65 | 8.81 |
| | garlic solution | | | | |
| 3 | 7% Streptococcus thermophilus fermented | 4.74 | 6.30 | 5.70 | 4.64 |
| | garlic solution | | | | |
| 4 | 10% Streptococcus thermophilus fermented | 4.74 | 4.40 | 3.70 | 0.00 |
| | garlic solution | | | | |

G. Fermented garlic solution (Leuconostoc lactis)

| | | | | | |
|---|---|---|---|---|---|
| Control | 0 | 5.54 | 7.70 | 8.30 | 9.36 |
| 1 | 3% Leuconostoc lactis fermented garlic solution | 5.54 | 5.18 | 5.48 | 7.48 |
| 2 | 5% Leuconostoc lactis fermented garlic solution | 5.54 | 5.29 | 4.74 | 6.00 |
| 3 | 7% Leuconostoc lactis fermented garlic solution | 5.54 | 5.18 | 4.18 | 0.00 |
| 4 | 10% Leuconostoc lactis fermented garlic solution | 5.54 | 5.30 | 4.48 | 0.00 |

As shown in Table A, the culture solution of Weissella koreensis snows a very low antibacterial effect against Candida albicans KCTC 7270 even at the concentration of 10 vol. %, and thus no meaningful effect of cell death is observed. The non-fermented garlic solution exhibits antibacterial effect only at the concentration of 10 vol. %. However, the fermented garlic solution fermented by lactic acid bacteria according to an embodiment of the present invention exhibits considerably superior antibacterial effect compared with the culture solution of Weissella koreensis and the non-fermented garlic solution, and in particular, shows a complete antibacterial effect of 100% at the concentration of 5 vol. % or more, and more surely at 10 vol. % or more. From such results of Table 6, it is confirmed that the fermented garlic solution fermented by lactic acid bacteria according to an embodiment of the present invention exhibits considerably superior antibacterial effects than those of the culture solution of lactic acid bacteria and non-fermented garlic solution.

### Example 3-6

### Antibacterial Effect Against Antibiotic-Resistant Bacteria (S008, PE021, PS025)

S008 (Citrobacter werkmanii), PE021 (Citrobacter freund), and PS025 (Escherichia coli) were diluted with peptone water (16.1 g/L, pH 7.0, Merck) and inoculated in a nutrient broth (Difco) so that the final concentration was 10⁴ to 10⁶ cfu/ml. To the obtained mixture, the cell-containing fermented garlic solutions prepared in Example 2, which are fermented by Weissella koreensis KCTC 3621, were added at the concentration of lwt %, 5 wt %, and 10 wt %, respectively. As a positive control, 50 ppm of an antibiotic (oxytetracyclin) was used. The obtained mixtures were cultured at 37° C., and the numbers of living cells of S008 (Citrobacter werkmanii), PE021 (Citrobacter freund), and PS025 (Escherichia coli) were measured in nutrient agar at 0, 4, 8, and 24 hours.

The antibacterial effects against the antibiotic-resistant bacteria are shown in Table 7.

TABLE 7

| | Number of living cells | | | |
|---|---|---|---|---|
| | (log₁₀ CFU/mL) | | | |
| Solution (%) | 0 | 4 | 8 | 24 |

A. S008 (Citrobacter werkmanii)

| | | | | |
|---|---|---|---|---|
| Control (0%) | 4.72 | 9.26 | 9.38 | 9.41 |
| Positive Control (OTC 50 ppm) | 4.72 | 8.08 | 9.00 | 9.60 |
| Fermented garlic solution 1% | 4.72 | 8.23 | 8.63 | 9.34 |
| Fermented garlic solution 5% | 4.72 | 5.57 | 0.00 | 0.00 |
| Fermented garlic solution 10% | 4.72 | 4.85 | 0.00 | 0.00 |

B. PE021 (Citrobacter freund)

| | | | | |
|---|---|---|---|---|
| Control (0%) | 6.83 | 8.48 | 9.95 | 9.30 |
| Positive Control (OTC 50 ppm) | 6.83 | 6.54 | 8.48 | 9.48 |
| Fermented garlic solution 1% | 6.83 | 6.90 | 8.59 | 9.26 |
| Fermented garlic solution 5% | 6.83 | 6.09 | 3.18 | 4.93 |
| Fermented garlic solution 10% | 6.83 | 6.14 | 0.00 | 0.00 |

C. PS025 (Escherichia coli)

| | | | | |
|---|---|---|---|---|
| Control (0%) | 4.65 | 7.65 | 9.11 | 9.34 |
| Positive Control (OTC 50 ppm) | 4.65 | 8.10 | 9.26 | 9.21 |
| Fermented garlic solution 1% | 4.65 | 5.08 | 6.97 | 9.10 |
| Fermented garlic solution 5% | 4.65 | 0.00 | 0.00 | 0.00 |
| Fermented garlic solution 10% | 4.65 | 0.00 | 0.00 | 0.00 |

As shown in Table 7, the fermented garlic solution fermented by lactic acid bacteria according to an embodiment of the present invention shows a complete antibacterial effect of 100% against the antibiotic-resistant bacteria at the concentration of 5 vol. % or more, while no antibacterial effect by the antibiotic was observed. From such results of Table 6, it is confirmed that the fermented garlic solution fermented by lactic acid bacteria according to an embodiment of the present invention exhibits excellent antibacterial effects against antibiotic-resistant bacteria.

### Comparative Example

Antibacterial Effects of Cell-Containing Lactic Acid Bacteria Culture in MRS Medium, Cell-Free Lactic Acid Bacteria Culture in MRS Medium, and Fermented Garlic Solution

To measure the antibacterial effects of a cell-containing lactic acid bacteria culture in a MRS medium, a cell-free lactic acid bacteria culture in a MRS medium, and a fermented garlic solution, a gram-positive bacterium, Staphylococcus aureus (KCTC 1621), a gram-negative bacterium, Escherichia coli (KCTC 2441), and a yeast, Candida albicans (KCTC 7270), were diluted with peptone water (16.1 g/L, pH 7.0, Merck) and inoculated in a nutrient broth (Difco) so that the final concentration was 10⁴ to 10⁶ cfu/ml. The lactic acid bacteria, such as Weissella koreensis KCTC 3621, Lactococcus lactis spp. lactis KCTC 3769, and Leuconostoc lactis KCTC 3528, to which the fermented garlic solutions of the present invention show particularly excellent antibacterial effects in Examples 3-1 to 3-6, were selected to be used in this example. The cell-containing lactic acid bacteria culture and the cell-free lactic acid bacteria culture were diluted with the nutrient broth (Difco) to the concentration of 0 vol. % (control), 3 vol. %, 5 vol. %, 7 vol. %, and 10 vol. %, respectively, and incubated at 37° C. The number of living cells was then measured in nutrient agar (Difco) at 0, 8, and 24 hours. As the cell-containing lactic acid bacteria culture, the cultures of Weissella koreensis KCTC 3621, Lactococcus lactis spp. lactis KCTC 3769, and Leuconostoc lactis KCTC 3528 according to Example 1 were used. A cell-free lactic acid bacteria culture was prepared by centrifuging the cell-containing lactic acid bacteria cultures at 15,000 rpm for 10 minutes, and filtering the supernatants with a 0.2 μm polypropylene syringe filter.

The antibacterial effects of the cell-containing lactic acid bacteria cultures and the cell-free lactic acid bacteria cultures are shown in Table 8.

TABLE 8

| | | Number of living cells | | |
|---|---|---|---|---|
| | | (log₁₀ CFU/mL) | | |
| Sample | Solution | 0 | 8 | 24 |

A. Staphylococcus aureus, KCTC 1621
a. Cell-containing culture solution (Weissella koreensis)

| | | | | |
|---|---|---|---|---|
| Control | 0 | 6.60 | 8.18 | 8.93 |
| 1 | 3% W. k cell-containing culture solution | 6.60 | 8.40 | 8.60 |
| 2 | 5% W. k cell-containing culture solution | 6.60 | 9.00 | 8.85 |
| 3 | 7% W. k cell-containing culture solution | 6.60 | 8.00 | 9.11 |
| 4 | 10% W. k cell-containing culture solution | 6.60 | 8.93 | 9.26 |

b. Cell-containing culture solution (Lactococcus lactis spp. lactis)

| | | | | |
|---|---|---|---|---|
| Control | 0 | 6.60 | 8.18 | 8.93 |
| 1 | Lactococcus lactis cell-containing culture solution 3% | 6.60 | 7.30 | 8.74 |
| 2 | Lactococcus lactis cell-containing culture solution 5% | 6.60 | 5.40 | 7.30 |
| 3 | Lactococcus lactis cell-containing culture solution 7% | 6.60 | 6.00 | 7.88 |
| 4 | Lactococcus lactis cell-containing culture solution 10% | 6.60 | 5.70 | 8.18 |

c. Cell-containing culture solution (Leuconostoc lactis)

| | | | | |
|---|---|---|---|---|
| Control | 0 | 6.60 | 8.18 | 8.93 |
| 1 | Leuconostoc lactis cell-containing culture solution 3% | 6.60 | 6.65 | 9.64 |
| 2 | Leuconostoc lactis cell-containing culture solution 5% | 6.60 | 6.30 | 8.70 |
| 3 | Leuconostoc lactis cell-containing culture solution 7% | 6.60 | 5.30 | 7.30 |
| 4 | Leuconostoc lactis cell-containing culture solution 10% | 6.60 | 4.70 | 3.70 |

d. Cell-free culture solution (Lactococcus lactis spp. lactis)

| | | | | |
|---|---|---|---|---|
| Control | 0 | 6.60 | 8.18 | 8.93 |
| 1 | Lactococcus lactis cell-free culture solution 3% | 6.60 | 8.00 | 8.30 |
| 2 | Lactococcus lactis cell-free culture solution 5% | 6.60 | 5.00 | 8.80 |
| 3 | Lactococcus lactis cell-free culture solution 7% | 6.60 | 4.00 | 5.70 |
| 4 | Lactococcus lactis cell-free culture solution 10% | 6.60 | 4.70 | 0.00 |

e. Cell-free culture solution (Leuconostoc lactis)

| | | | | |
|---|---|---|---|---|
| Control | 0 | 6.60 | 8.18 | 8.93 |
| 1 | Leuconostoc lactis cell-free culture solution 3% | 6.60 | 6.30 | 7.81 |
| 2 | Leuconostoc lactis cell-free culture solution 5% | 6.60 | 6.74 | 7.54 |
| 3 | Leuconostoc lactis cell-free culture solution 7% | 6.60 | 4.78 | 5.65 |
| 4 | Leuconostoc lactis cell-free culture solution 10% | 6.60 | 4.70 | 8.00 |

B. Escherichia coli. KCTC 2441
a. Cell-containing culture solution (Weissella koreensis)

| | | | | |
|---|---|---|---|---|
| Control | 0 | 8.65 | 9.19 | 9.54 |
| 1 | W. k cell-containing culture solution 3% | 8.65 | 9.48 | 9.34 |
| 2 | W. k cell-containing culture solution 5% | 8.65 | 9.42 | 9.41 |
| 3 | W. k cell-containing culture solution 7% | 8.65 | 3.70 | 9.28 |
| 4 | W. k cell-containing culture solution 10% | 8.65 | 9.53 | 9.40 |

b. Cell-containing culture solution (Lactococcus lactis spp. lactis)

| | | | | |
|---|---|---|---|---|
| Control | 0 | 8.65 | 9.19 | 9.54 |
| 1 | Lactococcus lactis cell-containing culture solution 3% | 8.65 | 8.40 | 9.93 |
| 2 | Lactococcus lactis cell-containing culture solution 5% | 8.65 | 9.08 | 9.88 |
| 3 | Lactococcus lactis cell-containing culture solution 7% | 8.65 | 6.54 | 8.93 |
| 4 | Lactococcus lactis cell-containing culture solution 10% | 8.65 | 5.18 | 5.54 |

c. Cell-containing culture solution (Leuconostoc lactis)

| | | | | |
|---|---|---|---|---|
| Control | 0 | 8.65 | 9.19 | 9.54 |
| 1 | Leuconostoc lactis cell-containing culture solution 3% | 8.65 | 8.81 | 9.53 |
| 2 | Leuconostoc lactis cell-containing culture solution 5% | 8.65 | 8.00 | 9.74 |
| 3 | Leuconostoc lactis cell-containing culture solution 7% | 8.65 | 6.48 | 9.53 |
| 4 | Leuconostoc lactis cell-containing culture solution 10% | 8.65 | 4.00 | 8.30 |

d. Cell-free culture solution (Lactococcus lactis spp. lactis)

| | | | | |
|---|---|---|---|---|
| Control | 0 | 8.65 | 9.19 | 9.54 |
| 1 | Lactococcus lactis cell-free culture solution 3% | 8.65 | 9.42 | 9.60 |
| 2 | Lactococcus lactis cell-free culture solution 5% | 8.65 | 7.70 | 9.49 |
| 3 | Lactococcus lactis cell-free culture solution 7% | 8.65 | 6.88 | 9.55 |
| 4 | Lactococcus lactis cell-free culture solution 10% | 8.65 | 5.18 | 0.00 |

e. Cell-free culture solution (Leuconostoc lactis)

| | | | | |
|---|---|---|---|---|
| Control | 0 | 8.65 | 9.19 | 9.54 |
| 1 | Leuconostoc lactis cell-free culture solution 3% | 8.65 | 9.52 | 9.53 |
| 2 | Leuconostoc lactis cell-free culture solution 5% | 8.65 | 6.34 | 9.30 |
| 3 | Leuconostoc lactis cell-free culture solution 7% | 8.65 | 6.23 | 0.00 |
| 4 | Leuconostoc lactis cell-free culture solution 10% | 8.65 | 4.00 | 0.00 |

C. Candida albicans, KCTC 7270
a. Cell-containing culture solution (Weissella koreensis)

| | | | | |
|---|---|---|---|---|
| Control | 0 | 8.65 | 9.08 | 9.53 |
| 1 | W. k cell-containing culture solution 3% | 8.65 | 9.34 | 9.40 |
| 2 | W. k cell-containing culture solution 5% | 8.65 | 9.28 | 9.27 |
| 3 | W. k cell-containing culture solution 7% | 8.65 | 9.34 | 9.37 |
| 4 | W. k cell-containing culture solution 10% | 8.65 | 9.41 | 9.37 |

b. Cell-containing culture solution (Lactococcus lactis spp. lactis)

| | | | | |
|---|---|---|---|---|
| Control | 0 | 8.65 | 9.08 | 9.53 |
| 1 | Lactococcus lactis cell-containing culture solution 3% | 8.65 | 8.48 | 9.82 |
| 2 | Lactococcus lactis cell-containing culture solution 5% | 8.65 | 7.70 | 9.02 |
| 3 | Lactococcus lactis cell-containing culture solution 7% | 8.65 | 5.70 | 8.00 |
| 4 | Lactococcus lactis cell-containing culture solution 10% | 8.65 | 4.70 | 7.40 |

c. Cell-containing culture solution (Leuconostoc lactis)

| | | | | |
|---|---|---|---|---|
| Control | 0 | 8.65 | 9.08 | 9.53 |
| 1 | Leuconostoc lactis cell-containing culture solution 3% | 8.65 | 8.93 | 9.60 |
| 2 | Leuconostoc lactis cell-containing culture solution 5% | 8.65 | 9.13 | 9.29 |
| 3 | Leuconostoc lactis cell-containing culture solution 7% | 8.65 | 4.30 | 6.74 |
| 4 | Leuconostoc lactis cell-containing culture solution 10% | 8.65 | 5.18 | 6.00 |

d. Cell-free culture solution (Lactococcus lactis spp. lactis)

| | | | | |
|---|---|---|---|---|
| Control | 0 | 8.65 | 9.08 | 9.53 |
| 1 | Lactococcus lactis cell-free culture solution 3% | 8.65 | 9.29 | 9.41 |
| 2 | Lactococcus lactis cell-free culture solution 5% | 8.65 | 9.46 | 9.57 |
| 3 | Lactococcus lactis cell-free culture solution 7% | 8.65 | 9.33 | 9.47 |
| 4 | Lactococcus lactis cell-free culture solution 10% | 8.65 | 3.65 | 7.70 |

e. Cell-free culture solution (Leuconostoc lactis)

| | | | | |
|---|---|---|---|---|
| Control | 0 | 8.65 | 9.08 | 9.53 |
| 1 | Leuconostoc lactis cell-free culture solution 3% | 8.65 | 9.30 | 7.81 |
| 2 | Leuconostoc lactis cell-free culture solution 5% | 8.65 | 9.35 | 7.54 |
| 3 | Leuconostoc lactis cell-free culture solution 7% | 8.65 | 6.18 | 5.65 |
| 4 | Leuconostoc lactis cell-free culture solution 10% | 8.65 | 5.30 | 8.00 |

As shown in Table 8, the cell-containing culture solution and the cell-free culture solution of lactic acid bacteria exhibit no antibacterial effects against Escherichia coli, KCTC 2441, or a weak antibacterial effect in which only a small number of bacteria Escherichia coli, KCTC 2441 are dead, except the cell-free culture solution of Lactococcus lactis spp. lactis and the cell-free culture solution of Leuconostoc lactis. From such results, the antibacterial effect of the fermented garlic solution fermented by lactic acid bacteria is considerably superior to those of cell-containing culture solution and the cell-free culture solution of the same lactic acid bacteria.

### Example 4

### Toxicity to Animal Cells of Non-Fermented Garlic and Fermented Garlic Solution Fermented by Lactic Acid Bacteria

A mixture of 10³ to 10⁴ of Madin-Darby canine kidney cells (MDCK cells, the medical college of Dankook University, Korea) and a media containing 10% FBS and 1% antibiotic (Penicillin G and Streptomycin, Sigma) were added to a 96 well plate to the amount of 100 μl per well, and cultured in an incubator (5% CO₂) at 37° C. for 24 hours. Then, the media was removed and each of 0, 1/2, 1/4, 1/8, 1/16, 1/32, 1/64, and 1/128 diluted samples (garlic solution of 100 g of ground garlic mixed with 100 g of water, and cell-containing fermented garlic solution fermented by Weissella koreensis prepared in Example 2) was added thereto in the amount of 100 μl. The mixtures were cultured in an incubator (5% CO₂) at 37° C. for 36 hours. MTT (5 mg/mL) was added to the wells in the amount of 50 μl per well (wherein 50 μl of media was further added to make the total volume 100 μl), and cultured in an incubator (5% CO₂) at 37° C. The MTT solution was removed, and a mixture solution of DMSO-ethanol (4:1) was added thereto in the amount of 100 μl per well to extract formazan therefrom, and the absorbance was measured using a microplate reader at 550 nm. The average of values obtained from 3 experiments was used, and the viability was determined by comparing with the initial cell concentration.

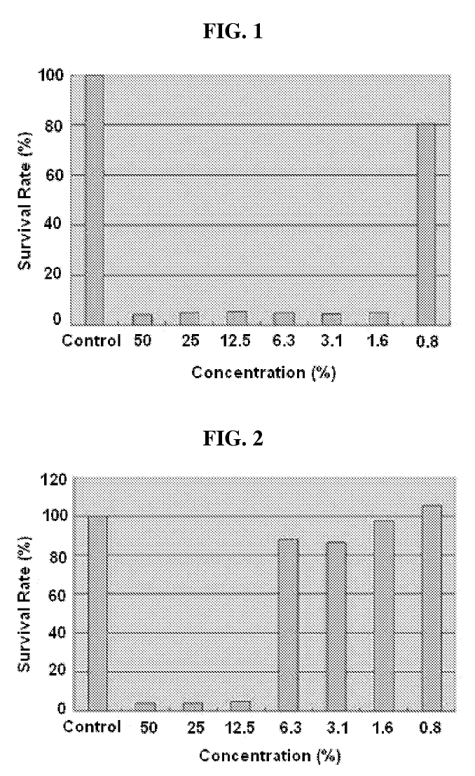

The viabilities of MDCK cells depending on the added concentration of the non-fermented garlic solution are shown in FIG. 1. The viabilities of MDCK cells according to the added concentration of the fermented garlic solution are shown in FIG. 2. As known from FIGS. 1 and 2, the fermented garlic solution exhibits low cytotoxicity that is 10 times lower than that of non-fermented garlic solution

1. An antibacterial agent comprising one or more selected from the group consisting of a fermented garlic solution fermented by lactic acid bacteria, and a concentrate and a dried substance thereof as an active ingredient.

2. The antibacterial agent according to claim 1, wherein said lactic acid bacteria is bacteria belonging to genera Weissella.

3. The antibacterial agent according to claim 2, wherein:
said lactic acid bacteria belonging to the genus Weissella is Weissella Koreensis.

4. The antibacterial agent according to claim 3, wherein said lactic acid bacteria is Weissella koreensis KCTC 3621.

5. The antibacterial agent according to claim 1, wherein said antibacterial agent has an antibacterial effect against gram-negative bacteria.

6. The antibacterial agent according to claim 5, wherein:
said gram-negative bacteria is Escherichia coli.

7. The antibacterial agent according to claim 1, wherein said fermented garlic solution is obtained by fermenting garlic with lactic acid bacteria, in the form of a cell-containing and/or cell-free solution.

8. A composition preventing bacterial infection comprising the antibacterial agent according to claim 1 as an active ingredient.

9. The composition according to claim 8, wherein said composition is provided in a form of feed.

10. A method for preventing bacterial infection, which comprises a step of applying an antibacterial agent comprising one or more selected from the group consisting of a fermented garlic solution fermented by lactic acid bacteria, and a concentrate and a dried substance thereof as an active ingredient according to claim 1, to an animal.

11. The method according to claim 10, wherein said lactic acid bacteria is one or more selected from the group consisting of bacteria belonging to genera Weissella, Lactobacillus, Lactococcus, Enterococcus, Streptococcus, and Leuconostoc.

12. The method according to claim 11, wherein:
said lactic acid bacteria belonging to genus Weissella is one or more selected from the group consisting of Weissella Koreensis, Weissella kimchii, Weissella cibaria, and Weissella confuse;
said lactic acid bacteria belonging to the genus Lactobacillus is one or more selected from the group consisting of Lactobacillus fermentum, Lactobacillus acidophilus, Lactobacillus delbrueckii spp. bulgaricus, Lactobacillus plantarum, Lactobacillus casei, and Lactobacillus reuteri;
said lactic acid bacteria belonging to the genus Lactococcus is one or more selected from the group consisting of Lactococcus lactis spp. lactis and Lactococcus lactis spp. Cremoris;
said lactic acid bacteria belonging to the genus Enterococcus is one or more selected from the group consisting of Enterococcus faecium and Enterococcus faecalis;
said lactic acid bacteria belonging to the genus Streptococcus is Streptococcus thermophilus; and
said lactic acid bacteria belonging to the genus Leuconostoc is one or more selected from the group consisting of Leuconostoc lactis, Leuconostoc citreum, and Leuconostoc mesenteroides.

13. The method according to claim 12, wherein said lactic acid bacteria is one or more selected from the group consisting of Weissella koreensis KCTC 3621, Lactobacillus fermentum KCTC 3112, Lactobacillus acidophilus KCTC 3164, Lactobacillus delbrueckii spp. bulgaricus KCTC 3635, Lactococcus lactis spp. lactis KCTC 3769, Enterococcus faecium KCTC 3122, Streptococcus thermophilus KCTC 3658, and Leuconostoc lactis KCTC 3528.

14. The method according to claim 10, wherein said antibacterial agent has an antibacterial effect against one or more selected from the group consisting of gram-positive and gram-negative bacteria, yeast, and antibiotic-resistant bacteria.

15. The method according to claim 14, wherein:
said gram-positive bacteria is one or more selected from the group consisting of Staphylococcus aureus, Streptococcus pneumoniae, Streptococcus pyogenes, and Enterobacter cloacae;
said gram-negative bacteria is one or more selected from the group consisting of Escherichia coli, Klebsiella pneumoniae, Proteus mirabilis, Haemophilus parainfluenzae, and Pseudomonas aeruginosa;
said yeast is one or more selected from the group consisting of Candida albicans, Candida glabrata, Candida krusei, and Cryptococcus neoformans; and
said antibiotic-resistant bacteria is a strain selected for its antibiotic-resistance to oxytetracycline from fecal excrement of poultry and swine.

16. The method according to claim 10, wherein said fermented garlic solution is obtained by fermenting garlic with lactic acid bacteria, in the form of a cell-containing and/or cell-free broth.
